(19) United States
(12) Patent Application Publication
(10) Pub. No.: US 2023/0346472 A1
(43) Pub. Date: Nov. 2, 2023

(54) Apparatus and Systems for Liquid Metal-Based Tunable Coaxial Antenna for Microwave Ablation

(71) Applicant: The Regents of the University of California, Oakland, CA (US)

(72) Inventors: Chih-Sheng Jason Chiang, Los Angeles, CA (US); Yahya Rahmat-Samii, Los Angeles, CA (US); Lingnan Song, Oakland, CA (US)

(73) Assignee: The Regents of the University of California, Oakland, CA (US)

(21) Appl. No.: 17/997,819

(22) PCT Filed: May 4, 2021
(86) PCT No.: PCT/US2021/030711

§ 371 Date: Nov. 2, 2022

Related U.S. Application Data
(60) Provisional application No. 63/019,920, filed on May 4, 2020.

(51) Int. Cl. A61B 18/18

(52) U.S. Cl. CPC A61B 18/1815; A61B 2018/1853; A61B 2018/1876; A61B 2017/0088

(57) ABSTRACT

Apparatus and systems of liquid metal-based tunable coaxial antenna for microwave ablation are described. Tunable antennas are capable of maintaining the optimal impedance matching in different organs of treatment as well as actively improving tissue heating during the course of the ablation. Efficient energy delivery to various tumor tissue and tissue conditions is critical in creating larger tumor margins and reducing the risk for local tumor progression. Efficient energy delivery can also help decrease the need for aggressive cooling mechanism from reflected power and antenna shaft heating. The tuning can be achieved by small physical motion of liquid metal plug actuated by pressure regulator.

### CROSS-REFERENCE TO RELATED APPLICATIONS

The current application claims the benefit of and priority under 35 U.S.C. § 119 (e) to U.S. Provisional Patent Application No. 63/019,920 entitled “Apparatus and Systems for Liquid Metal-Based Tunable Coaxial Antenna for Microwave Ablation” filed May 4, 2020. The disclosure of U.S. Provisional Patent Application No. 63/019,920 is hereby incorporated by reference in its entirety for all purposes.

### STATEMENT OF FEDERALLY SPONSORED RESEARCH

This invention was made with government support under grant number TR001881, awarded by the National Institutes of Health. The government has certain rights in the invention.

### FIELD OF THE INVENTION

The present invention generally relates to apparatus and systems for liquid-metal based tunable coaxial antenna; and more particularly to utilizing a liquid metal-based tunable coaxial antenna for microwave ablation.

### BACKGROUND OF THE INVENTION

Microwave ablation has emerged as a minimally invasive therapy for the treatment of various types of early-stage solid tumors. During microwave ablation, an interstitial antenna delivers high level microwave power from the generator to the target region, elevating local tissue environment to cytotoxic temperatures.

Liquid metals are commonly understood to be metal alloys with very low melting points which form a eutectic mixture that is liquid at room temperature and are typically electrically conductive. Liquid metals have traditionally been used in low power applications, on the order of 1-2 watts.

### BRIEF SUMMARY OF THE INVENTION

Apparatus and systems in accordance with various embodiments of the invention enable liquid metal-based tunable coaxial antennas for high-powered microwave ablation and other medical procedures. Liquid metal-based tunable antennas are able to change geometries of the antennas to match impedance of different organs and/or different states of tissue. In several embodiments, liquid metal-based tunable antennas can dynamically change antenna geometries during an ablation session to match the changing impedance of a target organ and/or tissue as it desiccates as a response to heat. In many embodiments, liquid metal tunable antennas can be used for microwave ablation in various organs and/or tissue. Examples of targeting organs in accordance with various embodiments of the invention include (but are not limited to): liver, lung, pancreas, spleen, adrenal gland, kidney, bladder, uterus, prostate, and/or bone. Examples of targeting soft tissue in accordance with various embodiments of the invention include (but are not limited to): skin, subcutaneous fat, muscle and lymph nodes. Different states of tissue can also be targeted using liquid metal tunable antennas in accordance with some embodiments. Examples of different tissue states in accordance with various embodiments of the invention include (but are not limited to): dehydrated, desiccated, and/or charred. In certain embodiments, antennas can be tuned for various conditions of lung including (but not limited to): different fraction of air within a phase of respiration of lung. In a number of embodiments, antennas can be tuned to localize within a particular lung lobe during microwave ablation. Many embodiments implement increased power delivery efficacy during microwave ablation with liquid metal-based tunable antennas. Such increased power delivery efficiency can reduce the need for large, cumbersome cooling apparatus, which can often further disrupt the heating efficiency of the antenna. Improved power delivery into tumor tissue can also provide better treatment outcomes and higher rate of disease free progression and overall survival in accordance with many various embodiments.

One embodiment of the invention includes a tunable monopole antenna comprising a coaxial cable comprising an inner conductor and an outer conductor, where a first length of one end of the coaxial cable has the outer conductor removed and the inner conductor exposed and the first length of the exposed inner conductor is not greater than an effective quarter wavelength (λ_{eff}/4) of the coaxial antenna; a first dielectric tube, wherein the first dielectric tube has a diameter and connects to the exposed inner conductor along a center of the coaxial cable, where the first dielectric tube covers a second length of the exposed inner conductor, a first part of the first dielectric tube is filled with a liquid metal, and a second part of the first dielectric tube is filled with a non-conductive oil, and the first dielectric tube is connected with a pressure regulator; a second dielectric tube, where the second dielectric tube has a diameter and connects to the first dielectric tube along a center of the first dielectric tube, where a first part of the second dielectric tube is filled with a liquid metal, and a second part of the second dielectric tube is filled with mineral oil; the length of the monopole antenna is from the exposed end of the outer conductor to the interface between the liquid metal and mineral oil of the second dielectric tube.

In a further embodiment, the length of the monopole antenna is tunable by applying a pressure to the pressure regulator.

In another embodiment, the length of the monopole antenna is tunable and the tunable length is from about 10 mm to about 16 mm.

In a still further embodiment, the length of the monopole antenna is tuned for an inflated lung and the length is about 15.5 mm.

In still another embodiment, an operating frequency of the tunable monopole antenna is from about 915 MHz to about 8GHz.

In a yet further embodiment, the operating frequency is about 2.45 GHz.

In yet another embodiment, an incident power of the tunable monopole antenna is from about 20 W to about 200 W.

In a further embodiment again, the incident power is about 50 W or about 100 W.

In another embodiment again, the second length of the exposed inner conductor covered by the first dielectric tube is smaller than the first length of the exposed inner conductor.

In a further additional embodiment, the diameter of the second dielectric tube is smaller than the diameter of the first dielectric tube.

In another additional embodiment, the diameter of the first dielectric tube is about 1/16 inch.

In a still yet further embodiment, the diameter of the second dielectric tube is about 1/32 inch.

In still yet another embodiment, the length of the first dielectric tube is about 8 mm.

In a still further embodiment again, the length of the second dielectric tube is about 8 mm.

In still another embodiment again, the dielectric tube comprises polytetrafluoroethylene.

In a still further additional embodiment, the liquid metal is a pressure-actuated eutectic liquid metal.

In a further embodiment again, the liquid metal comprises gallium-indium.

In yet another embodiment, the pressure regulator is a syringe.

In a yet further additional embodiment, the non-conductive oil is mineral oil.

A still yet another embodiment also includes a metallic tip.

In yet another embodiment, the metallic tip is fitted within a ceramic shaft.

Still another additional embodiment includes a method for performing microwave ablation comprising:
providing a power source;
providing a tunable monopole antenna comprising a coaxial cable comprising an inner conductor and an outer conductor, wherein a first length of one end of the coaxial cable has the outer conductor removed and the inner conductor exposed, where the first length of the exposed inner conductor is not greater than an effective quarter wavelength (λ_{eff}/4) of the coaxial antenna; a first dielectric tube, where the first dielectric tube has a diameter and connects to the exposed inner conductor along a center of the coaxial cable, the first dielectric tube covers a second length of the exposed inner conductor, a first part of the first dielectric tube is filled with a liquid metal, and a second part of the first dielectric tube is filled with a non-conductive oil, and the first dielectric tube is connected with a pressure regulator; a second dielectric tube, where the second dielectric tube has a diameter and connects to the first dielectric tube along a center of the first dielectric tube, a first part of the second dielectric tube is filled with a liquid metal, and a second part of the second dielectric tube is filled with mineral oil, and the length of the monopole antenna is from the exposed end of the outer conductor to the interface between the liquid metal and mineral oil of the second dielectric tube;
inserting the tunable monopole antenna to a target organ;
applying an incident power to the target organ, wherein the incident power has an operating frequency.

In a further embodiment, the length of the monopole antenna is tunable by applying a pressure to the pressure regulator.

In another embodiment, the length of the monopole antenna is tuned to match the target organ impedance during microwave ablation to lower reflect power.

In a still further embodiment, the length of the monopole antenna is tuned to match the impedance of a deflated lung, an inflated lung, or a liver.

In still another embodiment, the length of the monopole antenna is tunable and the tunable length is from about 10 mm to about 16 mm.

In a yet further embodiment, the length of the monopole antenna is tuned for an inflated lung and the length is about 15.5 mm.

In yet another embodiment, the operating frequency is from about 915 MHz to about 8GHz.

In another embodiment again, the operating frequency is about 2.45 GHz.

In a further additional embodiment, the incident power is from about 20 W to about 200 W.

In another additional embodiment, the incident power is about 50 W or about 100 W.

In a further additional embodiment, the second length of the exposed inner conductor covered by the first dielectric tube is smaller than the first length of the exposed inner conductor.

In another additional embodiment, the diameter of the second dielectric tube is smaller than the diameter of the first dielectric tube.

In a still yet further embodiment, the diameter of the first dielectric tube is about 1/16 inch.

In still yet another embodiment, the diameter of the second dielectric tube is about 1/32 inch.

In still another embodiment again, the length of the first dielectric tube is about 8 mm.

In a still further additional embodiment, the length of the second dielectric tube is about 8 mm.

In still another additional embodiment, the dielectric tube comprises polytetrafluoroethylene.

In yet another embodiment again, the liquid metal is a pressure-actuated eutectic liquid metal.

In a yet further embodiment, the liquid metal comprises gallium-indium.

In a further additional embodiment, the pressure regulator is a syringe.

In a still further embodiment, the non-conductive oil is mineral oil.

In still another embodiment again, the tunable monopole antenna further comprising a metallic tip.

In yet another embodiment, the metallic tip is fitted within a ceramic shaft.

Additional embodiments and features are set forth in part in the description that follows, and in part will become apparent to those skilled in the art upon examination of the specification or may be learned by the practice of the disclosure. A further understanding of the nature and advantages of the present disclosure may be realized by reference to the remaining portions of the specification and the drawings, which forms a part of this disclosure.

### BRIEF DESCRIPTION OF THE DRAWINGS

The description will be more fully understood with reference to the following figures, which are presented as exemplary embodiments of the invention and should not be construed as a complete recitation of the scope of the invention, wherein:

FIG. 1 illustrates a diagram of an image-guided microwave ablation for liver tumor treatment in accordance with prior art.

FIG. 2A illustrates the change in relative permittivity of lung and liver tissue as temperature reaches cytotoxic temperatures in accordance with prior art.

FIG. 2B illustrates the change in conductivity of lung and liver tissue as temperature reaches cytotoxic temperatures in accordance with prior art.

FIG. 3A illustrates a coaxial monopole antenna formed by stripping off a section of outer conductor at the end of coaxial cable by the effective quarter wavelength (λ_{eff}/4) for a specified coaxial monopole antenna in accordance with prior art.

FIG. 3B illustrates the effective quarter wavelength for various coaxial antennas consisting of an inner conductor wrapped with dielectric coating and inserted into lossy dielectric medium in accordance with certain embodiments.

FIGS. 4A-4D illustrate full-wave simulation comparing the reflection coefficient S₁₁ of organ-tuned antennas for inflated lung, deflated lung, bone, and normal liver tissue, respectively in accordance with certain embodiments.

FIG. 5A illustrates the effective quarter wavelength for three different coaxial antennas inserted into lossy dielectric medium in accordance with certain embodiments.

FIG. 5B illustrates the specific absorption rate mapping of a microwave ablation zone in an air-filled lung parenchyma using a lung tuned antenna and a liver tuned antenna in accordance with certain embodiments.

FIG. 6A illustrates a chart showing the reflection coefficient and aspect ratio as a function of increasing exposed dielectric length in accordance with certain embodiments.

FIG. 6B illustrates reflection coefficient as a function of operating frequency in accordance with certain embodiments.

FIGS. 7A and 7B illustrate simulations of the S parameter vs frequency for the 6 air content states utilizing the 13 mm and 15.5 mm monopole antenna respectively in accordance with certain embodiments.

FIGS. 8A and 8B illustrate reflected power at 2.45 GHz across organ-tuned antennas using the ex vivo ventilated porcine lung model at 50 W and 100 W respectively in accordance with certain embodiments.

FIGS. 9A and 9B illustrate microwave ablation size in lung tissue over a period of 1 minute and 5 minutes using 5 W and 100 W respectively in accordance with certain embodiments.

FIG. 10 illustrates schematics of a liquid metal tunable coaxial monopole antenna actuated by syringe with sample dimensions in accordance with embodiments.

FIG. 11 illustrates a liquid metal tunable antenna with cooling mechanism in accordance with certain embodiments.

FIG. 12 illustrates images of the liver tuned and lung tuned monopole antennas with exposed monopole lengths of 13 mm and 15.5 mm respectively in accordance with certain embodiments.

### DETAILED DESCRIPTION OF THE INVENTION

Turning now to the drawings, apparatus and systems for liquid metal-based tunable coaxial antenna for microwave ablation are described. In many embodiments, the tunable antennas can maintain optimal impedance matching among different organs of treatment, as well as during the ablation itself when the impedance is changing with respect to temperature. Examples of targeting organs in accordance with various embodiments of the invention include (but are not limited to): liver, lung, pancreas, spleen, adrenal gland, kidney, bladder, uterus, prostate, and/or bone. Antenna tuning according to many embodiments can be achieved by using liquid metal within a coaxial antenna. In some embodiments, liquid metal can be used in any part of a coaxial cable antenna that is electrically conductive. Examples of conductive parts of a coaxial cable in accordance with various embodiments of the invention include (but are not limited to): inner conductor, and/or outer conductor. In additional embodiments, the inner conductor can be attached to an electrically conductive material that can allow additional flexibility in impedance matching. In further embodiments, liquid metal can replace the outer conductor of a coaxial antenna and enable tuning by changing the length of the exposed dielectric. As can readily be appreciated, any of a variety of conductive parts of a coaxial cable can be implemented using liquid metal as appropriate to the requirements of specific applications in accordance with various embodiments. In several embodiments, liquid metal antenna tuning can be achieved by a pneumatic-driven physical displacement of liquid metal plug inside a dielectric tube interconnecting with the coaxial cable. Examples of pneumatic-driven approach in accordance with various embodiments of the invention include (but are not limited to): pneumatic pumps, and syringes. In some embodiments, liquid metal antenna tuning can be achieved by an electrical-driven physical displacement of liquid metal plug inside a dielectric tube interconnecting with the coaxial cable. Examples of electrical-driven approach in accordance with various embodiments of the invention include (but are not limited to): DC voltage differences applied through a carrier electrolyte—utilizing the effect of electro-wetting. Both pneumatic-driven and electrical-driven approaches can physically modify the antenna topology and frequency response. Many embodiments include increased efficacy of power delivery to tumor tissue during microwave ablation with liquid metal-based tunable antennas.

Some embodiments implement antenna tuning to match the impedance of a targeted organ and/or tissue. Different organs have intrinsic differences in impedance, giving a tunable antenna an advantage in heating over antennas with fixed geometries. Tissue impedance can also change with respect to temperature and desiccation. Examples of antenna tuning methods via pneumatic-driven or electrical-driven physical displacement of liquid metal in accordance with various embodiments of the invention include (but are not limited to): digital tuning, and/or analog tuning. As can readily be appreciated, any of a variety of tuning methods of an antenna can be utilized as appropriate to the requirements of specific applications in accordance with various embodiments.

In some embodiments, incorporation of liquid metal in a coaxial antenna can enable better power impedance matching during microwave ablation. A tunable antenna makes it possible to have impedance matching, leading to lower reflected powers when delivering high power into the tissue, in accordance with some embodiments. Conventionally, most semiconductor tuning is only performed at low power operation because otherwise it would enter a nonlinear region and performance would be suboptimal. Using liquid metal, antenna tuning for impedance matching can be done at higher power than previously utilized. Liquid metal has been implemented in a patch antenna to demonstrate lower loss, more power handling, and greater tuning range for wireless communications in a different environment than that discussed here with respect to various embodiments of the invention. (See, L. N. Song, et al., “Wideband Frequency Reconfigurable Patch Antenna With Switchable Slots Based on Liquid Metal and 3-D Printed Microfluidics”, IEEE Transactions on Antennas and Propagation, 67, 5, 2886-2895, 2019 May 4; the disclosure of which is herein incorporated by reference). However, the patch antenna incorporates liquid metal to sweep through various frequencies at low powers. This patch antenna utilized much lower power than would be used in ablation as discussed here. In comparison, many embodiments implement liquid metal for microwave ablation for tissue destruction and leverage the tuning portion to deliver levels of power that are magnitudes higher than that used in the patch antenna.

In several embodiments, a tunable antenna with a cooling mechanism is implemented. A metallic tip can be integrated into the antenna tip by fitting it within a ceramic shaft. The metallic tip and ceramic shaft can add to increase rigidity of the heating emission zone and to help add additional flexibility in impedance matching, in accordance with some embodiments. The cooling of tunable antennas can minimize coaxial cable shaft heating during occasions of impedance mismatches. As can readily be appreciated, the specific structures used to describe a tunable antenna in accordance with various embodiments of the invention are largely only limited by the requirements of specific applications.

### Microwave Ablation

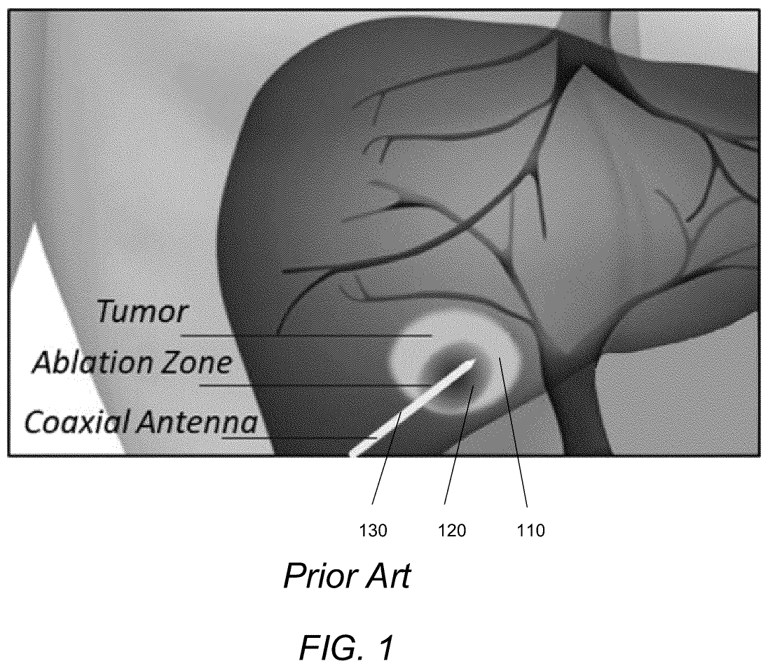

Microwave ablation has emerged as a minimally invasive therapy for the treatment of various types of early-stage tumors. During microwave ablation, an interstitial antenna can deliver high level microwave power from the generator to the target region, elevating local tissue environment to cytotoxic temperatures. (See, J. M. Bertram, et al., “A Review Of Coaxial-based Interstitial Antennas for Hepatic Microwave Ablation”, Critical Reviews™ in Biomedical Engineering, 34, 3, 2006; the disclosure of which is herein incorporated by reference). A schematic diagram of microwave ablation for liver tumor treatment is shown in FIG. 1. An interstitial antenna (130) can be inserted into the target tissue (110) to deliver high levels of microwave power from the generator and heat cancerous tissue to cytotoxic temperatures. Microwave ablation creates an ablation zone (120). Various organs and/or tissues can have different dielectric properties, resulting in different impedance matching conditions for designing the ablation antenna. Table 1 lists the dielectric properties for representative targeted organs (liver, lung, kidney, and bone), which can be used to design the antennas to optimally deliver energy into the targeted tissue and achieve temperatures in cytotoxic range.

TABLE 1
Dielectric properties for various organs
at 2.45 GHz and room temperature

| | Relative | | |
|---|---|---|---|
| Tissue | Permittivity | Conductivity (S/m) | Notes |
| Liver | 48-65 | 1.7 | Normal |
| Lung | 20.5 | 0.804 | Inflated |
| | 48.4 | 1.680 | Deflated |
| Kidney | 53 | 2.5 | |
| Bone | 10.3 | 0.46 | |

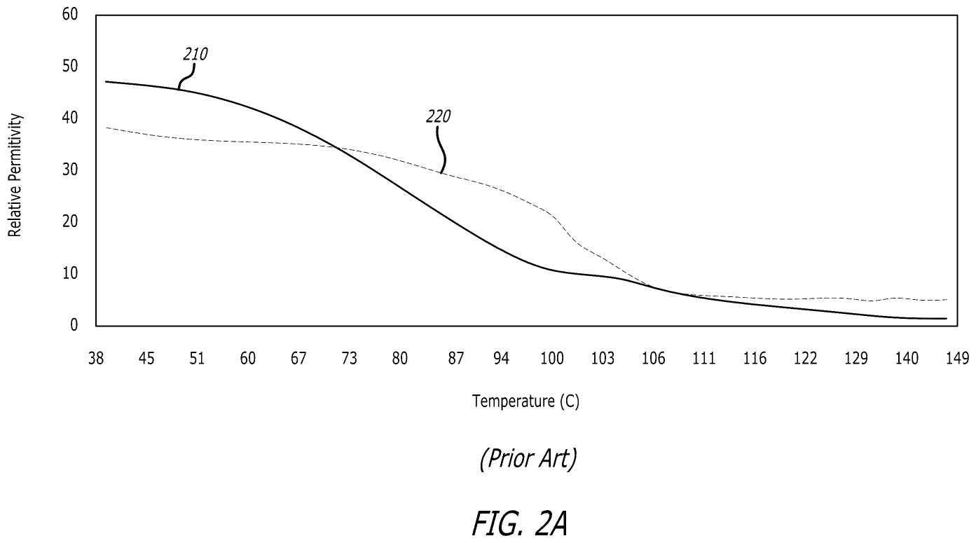

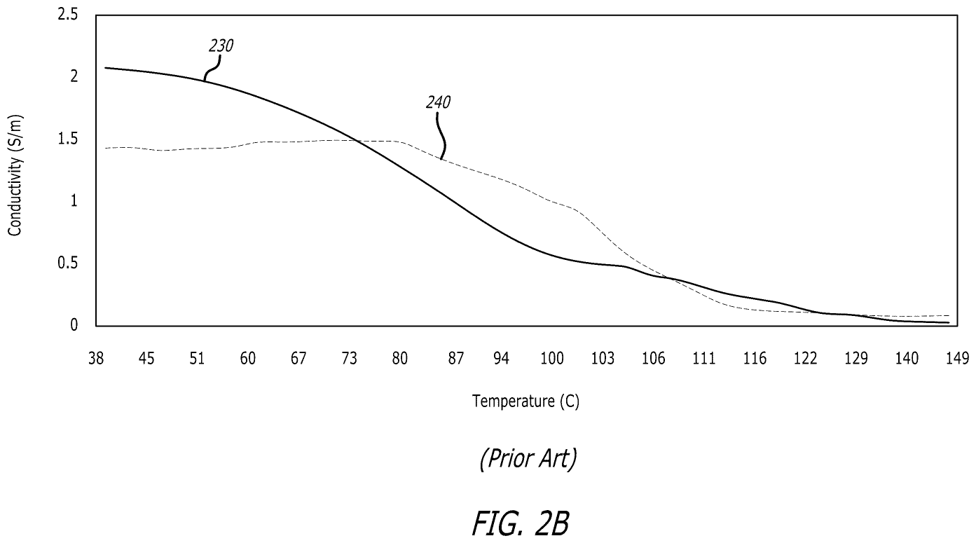

Many embodiments provide that microwave ablation can be used to treat tumors of the liver, pancreas, spleen, adrenal gland, kidney, bladder, uterus, prostate and lung. Each organ system can have different underlying electrical tissue properties, which can affect the efficient delivery of microwave power into the target tissue region due to the varying impedance matching conditions. The electrical properties of tissue also change with respect to temperature, which can add additional impedance mismatches during a single ablation session. Various organs and/or tissues can reach different dielectric properties including relative permittivity and conductivity at different temperatures. Many embodiments provide that antenna tuning can improve microwave ablation processes during the treatment by dynamically impedance matching as tissue reaches cytotoxic temperatures. FIGS. 2A and 2B illustrate temperature dependent relative permittivity and conductivity of lung and liver tissue. In FIG. 2A, the relative permittivity of lung tissue (220) decreases as the temperature rises from about 38° C. to about 149° C. The relative permittivity of liver tissue (210) decreases as the temperature rises from about 38° C. to about 149° C. In FIG. 2B, the conductivity of lung tissue (240) decreases as the temperature rises from about 38° C. to about 149° C. The conductivity of liver tissue (230) decreases as the temperature rises from about 38° C. to about 149° C. Many embodiments provide that the antenna design can be a critical element for the microwave ablation device and can have a significant impact on the ablation pattern and/or ablating power efficiency. Characteristic of ablation antennas in accordance with several embodiments of the invention can include: (a) small diameter for ease of insertion; (b) good impedance matching for efficient power transmission; (c) localized tumor-specific ablation pattern for accurate treatment; (d) integration into flexible coaxial cables (such as incorporating materials such as Gore-tex) that allow for endovascular or endobronchial guidance.

Organ-specific design of ablation antenna is conventionally implemented to achieve optimal performance and minimize the risk for incomplete therapy. Many embodiments implement coaxial antennas that can be adaptively tuned with liquid metal to maintain optimal impedance matching as it is transferred among different organs for treatment. Several embodiments include liquid metal coaxial antenna tuning to match impedance during the entirety of a single ablation session when the impedance mismatch arises from rising tissue temperatures. Some embodiments operate the tunable antenna at frequency ranges from MHz to GHz. Examples of tunable antenna operating frequency range in accordance with various embodiments of the invention include (but are not limited to): from about 915 MHz to about 8 GHz. For example, the operating frequency can be at about 2.45 GHz. As can readily be appreciated, any of a variety of operating frequency can be utilized as appropriate to the requirements of specific applications in accordance with various embodiments. Many embodiments provide that geometries of coaxial antennas can be tuned with liquid metal to match impedance of surrounding tissues and/or organs. Several embodiments provide simulations for a tunable coaxial monopole antenna within environments that mimic the electrical properties of various organs. In some embodiments, simulation results include environments of liver, bone and various states of lung tissue. As can readily be appreciated, any of a variety of targeting organ can be utilized as appropriate to the requirements of specific applications in accordance with various embodiments. Some embodiments compare reflection coefficients before and after tuning.

Apparatus and systems for microwave ablation with liquid metal-based tunable coaxial antenna in accordance with various embodiments of the invention are discussed further below.

### Tunable Coaxial Monopole Antenna for Optimal Impedance Matching in Different Tissue

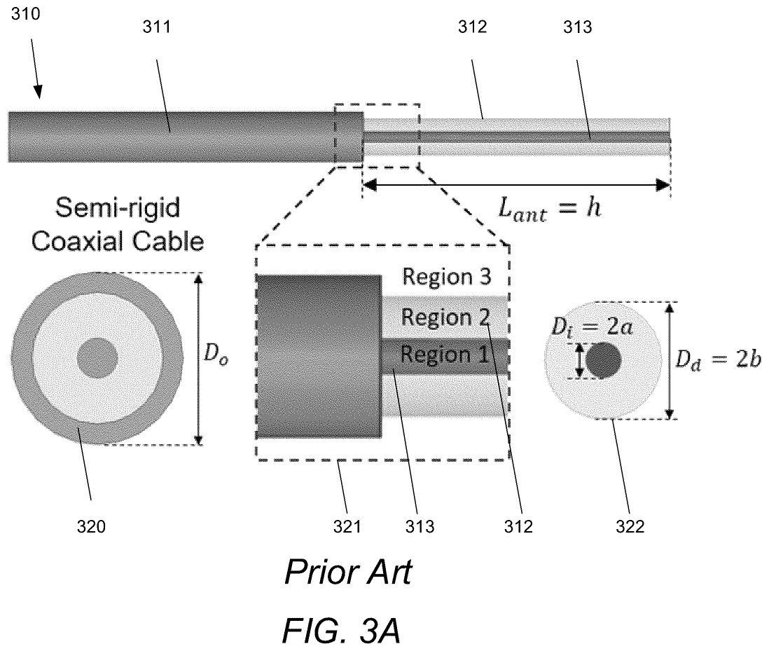

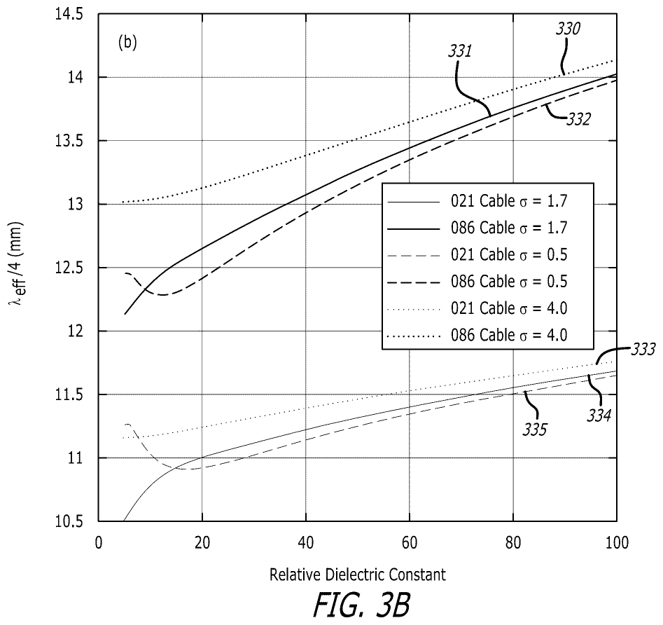

Many embodiments describe a tunable coaxial antenna that can match impedance in different tissue. Semi-rigid coaxial cables have been used as feeding transmission lines between power generator and applicator. In several embodiments, microwave ablation antennas can be integrated by modifying the geometry of the end of cable. In some embodiments, the monopole antenna can be formed by stripping or etching off a section of outer conductor at the end of coaxial cable by a length of Lₐₙₜ=λ_{eff}/4, where λ_{eff} represents the effective wavelength for the coaxial antenna. FIG. 3A illustrates the structure of a coaxial monopole antenna. A coaxial cable includes an outer conductor, a dielectric material, and an inner conductor. A coaxial monopole antenna (310) can be formed by stripping off a length of about quarter effective wavelength (λ_{eff}) of the outer conductor (311), where the dielectric material (312) and the inner conductor (313) of that length are exposed. The length of the monopole antenna Lₐₙₜ is h. The cross section of the coaxial cable (320) shows the diameter of the cable is Dₒ. A part of the coaxial monopole antenna (321) illustrates the inner conductor (313) as Region 1, the dielectric material (312) as Region 2, and the surround environment including tissue and/or organ as Region 3. The cross section of the coaxial monopole antenna (322) shows the diameter of the inner conductor Dᵢ=2a, and the diameter of the dielectric material D_{d}=2b. Table 2 lists the dimensions of coaxial cable in three standards as cable 086, cable 034, and cable 021.

TABLE 2
Dimensions of coaxial cable with the 086, 034, and 021 standards

| Cable | Dielectric ∈ᵣ | Dᵢ (mm) | D_{d} (mm) | Dₒ (mm) |
|---|---|---|---|---|
| 086 | 2.1 | 0.51 | 1.68 | 2.21 |
| 034 | 2.1 | 0.20 | 0.66 | 0.86 |
| 021 | 2.1 | 0.10 | 0.38 | 0.51 |

The effective quarter wavelength can be found analytically through the insulating antenna theory for an inner conductor wrapped with multiple dielectric layers and immersed into lossy dielectric medium. (See, R. W. King, et al., “The Electromagnetic Field Of An Insulated Antenna In A Conducting Or Dielectric Medium”, IEEE Transactions on Microwave Theory and Techniques, 31, 7, 574-583, 1983 July; the disclosure of which is herein incorporated by reference). The effective propagation constant k_{L} can be given by:

$${k}_{L} = {{k}_{2} [ \frac{ln ( \frac{b}{a} ) {k}_{3} {bH}_{1}^{( 1 )} ( {k}_{3} b ) + {H}_{0}^{( 1 )} ( {k}_{3} b )}{ln ( \frac{b}{a} ) {k}_{3} {bH}_{1}^{( 1 )} ( {k}_{3} b ) + \frac{{k}_{2}^{2}}{{k}_{3}^{2}} {H}_{0}^{( 1 )} ( {k}_{3} b )} ]}^{1 / 2} \qquad ( 1 )$$

where k₂ and k₃ denote the propagation constant in cable dielectric and lossy tissue medium, a and b denote the radius of inner conductor and cable dielectric. H⁽¹⁾ represents Hankel function of the first kind. The effective quarter wavelength can be computed with λ_{eff}/4=π/2k_{L}. The effective quarter wavelengths for the coaxial antenna including an inner conductor wrapped with dielectric coating and inserted into lossy dielectric medium can be plotted with varying tissue dielectric constant and conductivity. The effective quarter wavelengths for coaxial 086 and 021 cables are plotted with varying relative dielectric constant in accordance with an embodiment of the invention in FIG. 3B. The effective quarter wavelengths for 086 cable with conductivity σ=4.0 (330), 086 cable with conductivity σ=1.7 (331), and 086 cable with conductivity σ=0.5 (332) are plotted against relative dielectric constant from 0 to 100. The effective quarter wavelengths for 021 cable with conductivity σ=4.0 (333), 021 cable with conductivity σ=1.7 (334), and 021 cable with conductivity σ=0.5 (335) are plotted against relative dielectric constant from 0 to 100. FIG. 3B shows that at a relative dielectric constant around 40, the effective quarter wavelength can be around 13 mm for 086 cable.

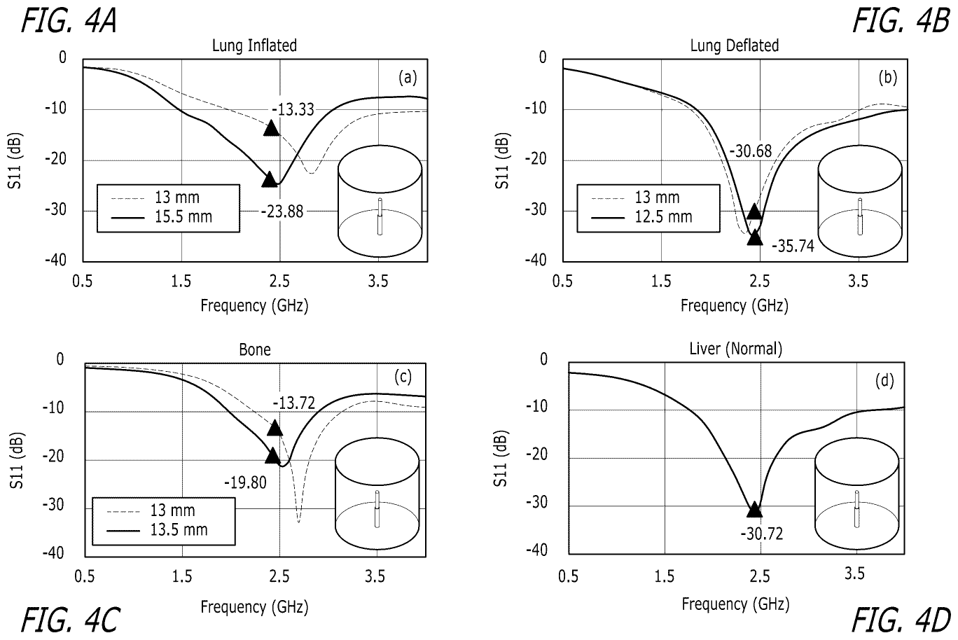

Many embodiments indicate the effective quarter wavelength may not be very sensitive to the variation of tissue dielectric property from the analytical solution, which varies within the range of ±1 mm for ϵᵣ from 10 to 90. However, the small deviation of impedance matching condition could result in a relatively large increase in the reflected power, due to the high power level of incident power in accordance with certain embodiments. The incident power can be delivered (but is not limited to) in the range of about 20 to about 200 W. As can readily be appreciated, any of a variety of power levels can be utilized as appropriate to the requirements of specific applications in accordance with various embodiments. In several embodiments, adaptive tuning for the coaxial antenna can be implemented to accommodate various tissue properties among different organs. FIG. 4A-4D plot the full-wave simulation results of S₁₁ with monopole length tuned for different tissue comparing with a fixed 13 mm monopole in accordance with an embodiment of the invention. Inflated and deflated lung tissue are illustrated in FIG. 4A and FIG. 4B. Bone tissue is illustrated in FIG. 4C, and normal liver tissue in FIG. 4D. The simulation model can be set up in high-frequency structure simulator (HFSS), with a coaxial monopole inserted into a cylindrical lossy dielectric box mimicking the surrounding tissue. The 13 mm effective quarter wavelength has been used in the traditional monopole antenna design for microwave ablation procedures. In many embodiments, the use of 13 mm monopole coaxial cable has been implemented via parametric methods, although an analytic approach has been outlined by equation (1). In several embodiments, at the operating frequency of about 2.45 GHz, improvement in S₁₁ can be observed. These improvements can be achieved with other microwave frequencies ranging from about 915 MHz to about 8 GHz. Some embodiments show improvements in S₁₁ of about 5 dB to about 10 dB, with minor tuning on the monopole length within a 3 mm range. These improvements can be applicable in creating sufficient ablation margin around a tumor, depending on the incident power.

Several embodiments implement antenna tuning to match impedance of a targeted organ and/or tissue. Examples of antenna tuning methods in accordance with various embodiments of the invention include (but are not limited to): digital tuning and analog tuning. In some embodiments, analog tuning of antenna may involve changing the actual geometry of the microwave antenna configuration. Several embodiments describe a movable outer cannula that can be moved to change the length of the exposed dielectric material. Some embodiments include pumping a liquid metal through the inner metal to the tip of the antenna such that it can provide a weighted load that can augment the impedance matching process. In some other embodiments, digital tuning methods can be adopted. Many embodiments use a matching circuit comprised of several discrete inductors and capacitors. The matching circuit may adjust the antenna output impedance to match the target biological tissue impedance to optimize heating.

While various processes for impedance matching using tunable coaxial antenna are described above with reference to FIG. 4A-4D, any of a variety of processes that utilize tunable coaxial antenna to match impedance of different tissue can be utilized in the design and/or fabrication of tunable antenna as appropriate to the requirements of specific applications in accordance with various embodiments of the invention.

### Lung-Tuned Monopole Antenna for Lung Cancer Treatment

Lung cancer remains a leading cause of cancer deaths among men and women in the United States, accounting for approximately 25% of all cancer deaths. While surgical lobectomy remains a standard care, only a fraction of the patients are considered eligible due to associated comorbidities such as poor pulmonary reserve, concomitant pulmonary fibrosis, or advanced frailty. Percutaneous image-guided thermal ablations can be an option due to their association with fewer complications, quicker recovery and minimal blood loss. (See, e.g., M. Venturini, et al., Cardio Vascular and Interventional Radiology, 2020, 1-17; the disclosure of which is incorporated herein by reference.) During thermal ablation procedures, energy is transmitted through an antenna or applicator under image guidance toward target tumor tissue. The ablation process continues until the tumor tissue is heated to cytotoxic temperatures with sufficient margins, all while minimizing damage to normal surrounding tissue. (See, e.g., M. Ahmed, et al., Journal of Vascular and Interventional Radiology, 2014, 11, 25, 1706-1708; the disclosure of which is incorporated herein by reference.) For treating lung malignancies, thermal ablations can be also associated minimal effect on pulmonary function, a side effect that can be seen in both radiation therapy or surgical resection.

Considerations when choosing ablation modalities include the size and anatomic location of the target tumors. Larger tumors are associated with more advanced cancer stages and higher rates of recurrence. For that reason, using microwave ablation has gained traction over radiofrequency ablation and cryoablation because of its ability to be tuned to the target tissue, delivering higher levels of power into the tissue and creating larger, more homogeneous ablation zone. (See, e.g., C. L., Brace, et al., Journal of Vascular and Interventional Radiology, 2010, 21, 8, 1280-1286; C. L. Brace, et al., Curr Probl Diagn Radiol, 2009, 38, 3, 135-143; the disclosures of which are incorporated herein by references.) In addition, when compared to radiofrequency ablation, microwave ablation has been associated with less postprocedural pain. Despite these advantages, the outcome data from microwave ablation remains heterogeneous due to variations in target patient population and follow up periods.

The general concept of tuning a microwave antenna toward a target tissue has been established in literatures. (See, e.g., R. D. Nevels, et al., IEEE Transactions on Biomedical Engineering, 1998, 45, 7, 885-890; S. Labonte, et al., IEEE Transactions on Microwave Theory and Techniques, 1996, 44, 10, 1832-1840; N. A. Durick, et al., Radiology, 2008, 247, 1, 80-87; C. L. Brace, et al., IEEE MTT-S International Microwave Symposium Digest, 2004, 1437-1440; S. Etoz, et al., International Journal of RF and Microwave Computer-Aided Engineering, 2018, 28, 3, p. e21224; J. M. Bertram, et al., Crit Reve Biomed Eng, 2006, 34, 3, 187-213; the disclosures of which are incorporated herein by references.) Reflected power may be a heavily weighted variable in antenna optimization strategies because it can be related to how well matched an antenna or transmission line is to the target organ. Low reflected power can be indicative of optimal impedance matching and translates into efficient power deposition into the tissue. Conversely, high reflected power can signify an impedance mismatch between the target tissue and antenna, leading to decreased power delivered into the tissue. A mismatch between the antenna and the tissue can also cause power to be reflected back into the generator and cause the formation of a standing wave into the feeding line, leading to excess heating loss along the shaft of the antenna. The other important metric in antenna optimization strategy is the shape of the ablation zone within the tissue. More spherical ablation zones may be desirable as it can encompass a round tumor with appropriate margins and minimize the risk for backward heating. Heat that spreads backward along the shaft of the antenna can damage the superficial surfaces of the organ, often times leading to post-procedural pain. In the lungs, inadvertent backward heating can lead to more severe complications such as pneumothorax or bronchopleural fistulas.

Validation studies for organ-tuned antennas have been performed on in vivo and ex vivo liver tissue. Within lung tissue, however, optimization and validation of lung tuned antennas currently remains in its nascency. Part of the reason is that lung tissue composition varies by gas exchange during respiration and anatomic location, with the central regions being more heterogeneous compared to peripheries. (See, e.g., R. A. Al-Hakim, et al., Journal of Vascular and Interventional Radiology, 2016, 27, 9, 1380-1386; the disclosure of which is incorporated herein by reference.) The validation component of lung-tuned antennas is also challenging in an ex vivo model where gas content needs to be maintained. As a result, current literature on the performance and clinical outcomes of lung ablations is limited and confounded by heterogeneous data.

Antenna tuning for microwave ablations in the lung remains an active area of investigation. A prior in vivo study of the lungs using an open thoracotomy approach has described a manually-tuned antenna using a triaxial design. (See, e.g., N. A. Durick, et al., Radiology, 2008, 247, 1, 80-87; the disclosure of which is incorporated herein by reference.) An accompanying follow up study also found positive results using lung-tuned triaxial antennas, with the microwave ablation probes were placed near the peripheries of the lungs where the lung parenchyma was more homogeneous. (See, e.g., C. L. Brace, et al., Current Problems in Diagnostic Radiology, 2009, 38, 3, 135-143; the disclosure of which is incorporated herein by reference.) There has been increasing interest in also implementing microwave antenna ablations into flexible antennas for the purpose of bronchoscopically guided ablations. (See, e.g., H. B. Yuan, et al., Translational Lung Cancer Research, 2019, 8, 6, 787-796; J. Ferguson, et al., CHEST, 2013, 144, 4, 87A; the disclosures of which are incorporated herein by references.) Characterizing temperature-dependent dielectric properties of lung have only begun being investigated, although varying air content within the lung remains unaccounted for. (See, e.g., J. Sebek, et al., Medical Physics, 2019, 46, 10, 4291-4303; the disclosure of which is incorporated herein by reference.) The dielectric properties of a decompressed lung approach that of the liver, which can be expected given that a decompressed lung has minimal air content. In contrast, many embodiments utilize a fully expanded lung model to better match the known dielectric and conductivity of a fully inflated lung. However, the fully expanded lung also likely represents the upper limits of performance for a lung-tuned antenna given that there will be increasing load mismatches that occur during lung decompression. Several embodiments provide that additional ex vivo validations for lung-tuned antenna design can benefit from using ventilator-controlled models, which can control the phase of respiratory cycle during which the ablation is performed.

Many antenna designs have been investigated for the purposes of improving load matching and localized heating to the antenna tip. King et al. developed the first analytical solution and derived the radiation fields of a multi-section insulated antenna in a conductive media. (See, R. W. King, et al., IEEE Transactions on Microwave Theory and Techniques, 31, 7, 574-583, 1983; the disclosure of which is herein incorporated by reference). This solution was expanded to interstitial monopole and dipole antennas for hyperthermia treatment. (See, e.g., M. F. Iskander, et al., IEEE Trans Biomed Eng., 1989, 36, 2, 236-46; P.C. Cherry, et al., IEEE Trans Biomed Eng., 1993, 40, 8, 771-9; the disclosures of which are incorporated herein by references.) Early antenna designs focused on optimized matching to the tissue load and localized heating pattern but its energy delivery efficiency was highly dependent on insertion depth and there was high amounts of backward heating along the antenna shaft from power loss. A well-known alternative to reduce backward heating problem independent of load matching was to electrically connect a metallic choke to the antenna's outer conductor to minimize axial current flow and localize power deposition near the tip of the antenna. The cap choke antenna was described using an annular cap and a coaxial cap in a coaxial antenna—together utilized to match an antenna to a coaxial transmission line. (See, e.g., S. Pisa, et al., IEEE Trans Biomed Eng., 2001, 48, 5, 599-601; J. C. Lin, et al., IEEE Trans Biomed Eng., 1996, 43, 6, 657-660; the disclosures of which are incorporated herein by references.) This cap choke design minimized SAR at the insertion point of the catheter antenna and minimized the amount of reflected power up the transmission line. The cap choked antenna design also allowed energy to be delivered into the tissue without being affected by the depth of insertion. A floating sleeve microwave antenna, offered another approach by utilizing a metal conductor that was electrically isolated from the outer connector of the antenna coaxial body, creating a highly localized heating zone independent of insertion depth. (See, D. Yang, et al., IEEE Trans Biomed Eng., 2006, 53, 3, 533-7; the disclosure of which is herein incorporated by reference). The floating sleeve prevented backward heating of the antenna, while creating an efficient ablation zone in the liver. However, many of these designs required a large diameter on the order of 6 or 7 gauge that often precluded its use in percutaneous therapies. Other alternatives to using a choke design to minimize backward heating is to utilize slot antennas. Slot antennas are capable of actively localizing the heating zone to the tip and is also not affected by insertion depth, utilizing overlapping surface currents from each slot. (See, K. Sato, et al., IEEE Trans Microwave Theory and Techniques, 2000, 48, 1800-1806; the disclosure of which is herein incorporated by reference). Highly resonant antennas such as the triaxial antenna have also been utilized that can be fabricated with thinner, less invasive designs, and do not require the outer conductor to be connected to the antenna itself. (See, C. L. Brace, et al., IEEE MTT-S International Microwave Symposium Digest, 2004, 3, 1437-1440; the disclosure of which is herein incorporated by reference).

Many embodiments provide analytical approaches to fabricate lung-tuned microwave antennas and the performance at conventional time-power settings that could treat small lesions but minimize the risk for shaft heating and pleural damage. The lung-tuned antennas in accordance with several embodiments are validated against liver-tuned antennas control in an ex vivo lung connected to a clinical-grade ventilator, keeping the lung inflated for the entirety of the ablation. The lung-tuned antennas are able to create significantly more spherical ablation zones at about 1 minute using about 50 W, as well as at about 5 minute at about 100 W. In some embodiments, lung tuned antennas can offer about 15-25% heating efficiency over liver-tuned antennas at the 1 minute time point with about 50 W and about 100 W, as well as the 5 minute time point with about 50 W.

Several embodiments implement the ex vivo ventilated lung model that may create a more accurate representation of the lungs compared to strictly deflated lung tissue. A fully deflated lung by itself has similar electrical properties as a liver and can be limited in validating lung-based cancer therapies. By connecting the main bronchus to a ventilator, air can be added into the lung parenchyma in a controlled setting and recapitulate the electrical and thermal properties of the lung. While adding ventilation alone does not account for blood perfusion or lymphatic architecture of the lung, the ventilator-controlled ex vivo model in accordance with some embodiments is a more accurate representation compare to completely-collapsed ex vivo models. An ex vivo ventilated lung model can provide a similarly controlled and accurate environment at a fraction of the cost and complexity of an in vivo porcine lung model.

In several embodiments, the amount of reflected power remains higher than expected for the duration of the ablation period. The reason for the mismatch may lie within the local anatomy of the central aspects of the lung. The lung parenchyma is increasingly heterogeneous centrally, which is made up of proportionally greater volumes of bronchioles, lymphatics and pulmonary vasculature. This is in contrast to the more peripheral regions of the lungs, which are more homogeneous, comprising primarily of air with a small fraction of microvasculature, alveoli and respiratory ducts. As a result, the anatomy in the peripheral lung fields appears homogeneous on even high-resolution CT imaging. Given the small size of the ex vivo lungs, the high reflection coefficient in accordance with certain embodiments may result from the lung-tuned antennas being placed more centrally and interfacing with a combination of large airways and pulmonary vasculature. Certain embodiments provide that lung-tuning microwave antennas should consider the higher contribution of blood flow and airways in the central lungs compared to the lung peripheries.

Some embodiments implement a parametric optimization function to balance the antenna reflection coefficient with the ablation aspect ratio, metrics that are useful for characterizing antenna performance. While there is a significantly larger aspect ratio using the lung-tuned antenna at the 50 W, 1 minute interval, there is not a significant difference between antennas at other power-time combinations. In several embodiments, there is a significant increase in aspect ratio when higher powers or higher time settings are utilized. Utilizing powers of about 100 W for about 5 minutes can lead to ablation zones with aspect ratios that average about 0.8, which is nearly spherical in shape. A number of embodiments provide that optimize lung ablation shapes should consider the amount of power that is being delivered to help better modulate the shape over time.

### Lung-Tuned Monopole Antenna for Microwave Ablation

Many embodiments provide lung-tuned monopole antennas to deliver microwave energy at about 2.45 GHz into a ventilator-controlled ex vivo lung model. Details about analytic and parametric approaches to create optimized monopole antennas that are impedance-matched to aerated lung tissue can be found in the Exemplary Embodiments section. In several embodiments, the lung-tuned antennas can be fabricated using copper 086 semi-rigid copper coaxial cable. In some embodiments, the lung-tuned antennas can be inserted centrally into lobes of an ex vivo porcine lung that is fully inflated to physiologically appropriate volumes. Microwave ablations can be created at about 50 W and about 100 W for about 1 minute and about 5 minutes in accordance with several embodiments. Many embodiments compare reflected power, cross sectional ablation sizes and spherical shape of the lung-tuned antennas against liver-tuned antennas in the ventilator controlled ex vivo lung tissue. Several embodiments provide that the lung tuned antennas can deliver energy more efficiently, with less reflected power. In some embodiments, lung tuned antennas exhibit reflected power of about 11.8±3.0 W, compared to the liver-tuned antennas of about 16.3±3.1 W, at about 50 W at about 1 minute. In certain embodiments, lung tuned antennas show reflected power of about 16.2±2.8 W, compared to the liver-tuned antennas of about 19.4±2.9 W at about 50 W at about 5 minutes. In a number of embodiments, lung tuned antennas show reflected power of about 29.0±3.5 W, compared to liver-tuned antennas of about 38.0±5.3 W at about 100 W at about 1 minute time point. Many embodiments compare overall ablation zone sizes between the lung tuned and liver tuned antennas. In several embodiments, the lung-tuned antenna can create a more spherical ablation zone compared to the liver-tuned antenna. At the 1 minute 50 W, aspect ratio of lung tuned antenna ablation zone is about (0.43±0.07), and the aspect ratio of liver tuned antenna ablation zone is about (0.38±0.04). In both antenna groups, there is a significant rise in the ablation zone aspect ratio between 1 and 5 minutes, indicating that higher power and time settings can increase the spherical shape of ablation zones when using tuned antennas. Many embodiments provide that the combined analytic and parametric antenna design approach can be implemented in adaptive tissue-tuning for real-time microwave ablation optimization in lung tissue.

Many embodiments provide optimized monopole antennas to transmit energy efficiently into the dielectric load of an air expanded lung while maintaining a spherical ablation zone. This lung-tuned antenna in accordance with several embodiments can be used to create microwave ablation zones in a ventilator-controlled ex vivo porcine lung model. Some embodiments provide analytic solutions to optimize monopole antenna designs that can efficiently deliver microwave energy into lung tissue. Details about analytic and parametric optimization of monopole antennas can be found in the Exemplary Embodiments section. A number of embodiments combine the analytic solution with a parametric approach to simultaneously create a spherical ablation zone within the lung while preserving heating efficiency. Many embodiments validate the optimized antenna designs utilizing ventilator controlled ex vivo lung models.

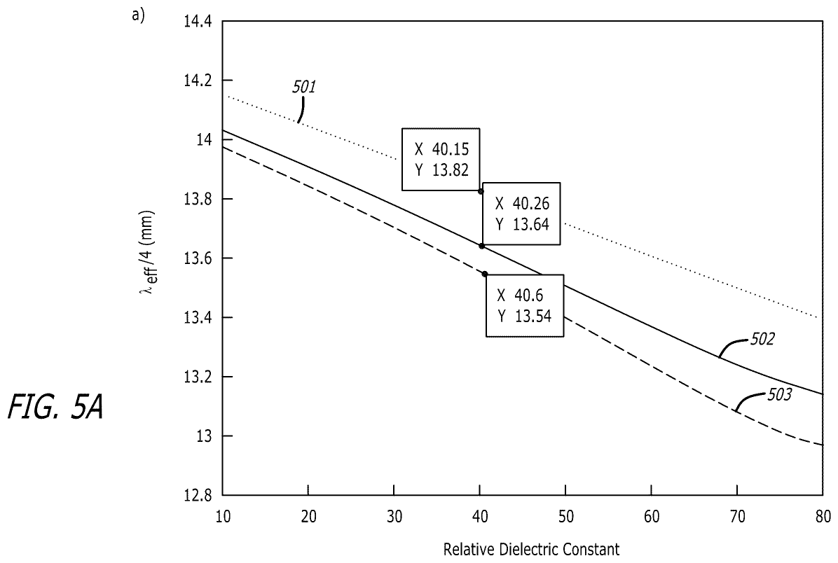

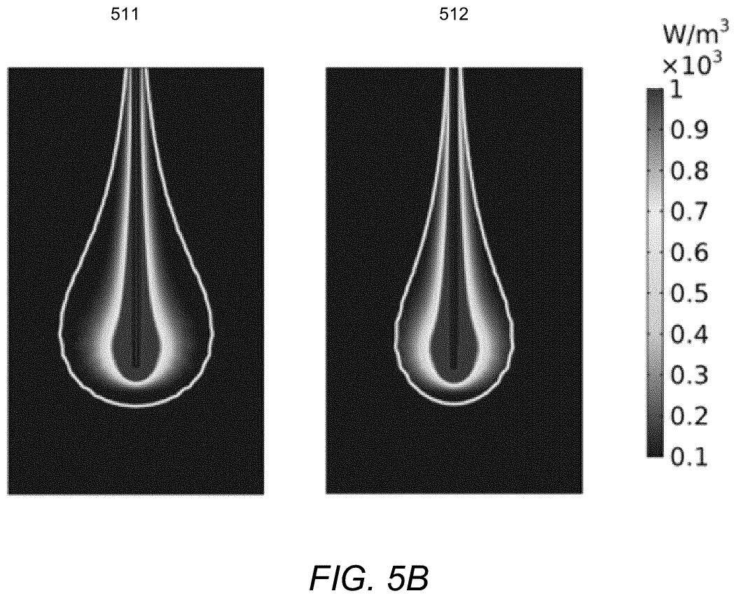

Many embodiments provide analytical modeling of monopole antenna designs. The effective quarter wavelength computed with λ_{eff}/4=π/2β_{eff} is plotted in FIG. 5A for 086 cable with varying tissue dielectric constant and conductivity in accordance with an embodiment of the invention. The effective quarter wavelength for the coaxial antenna consisting of an inner conductor wrapped with dielectric coating and inserted into lossy dielectric medium. The exposed length of the dielectric may be insensitive to large variations in conductivity and relative dielectric constant. Monopole antenna of 086 cable with σ=4 (501), 086 cable with σ=1.7 (502), and 086 cable with σ=0.5 (503) are shown in FIG. 5A. In an air-filled lung environment, an exposed dielectric length of about 15.5 mm may provide the optimal impedance match in accordance with some embodiments. Several embodiments provide that in a liver environment, an exposed dielectric length of about 13.0 mm is identified. In certain embodiments, the effective quarter wavelength for the lung may not be sensitive to the variation of tissue dielectric property, which varies within the range of ±1 mm for ϵᵣ from 10 to 90 and σᵣ from 4 to 0.5 S/m.

Specific absorption rate (SAR) mapping of a microwave ablation zone in an air-filled lung parenchyma using a lung tuned antenna and a liver-tuned antenna is illustrated in FIG. 5B in accordance with an embodiment of the invention. Lung tuned antenna in aerated lung tissue (511) and liver tuned antenna in aerated lung tissue (512) are both shown. The green isocontour line represents the 300 W/kg SAR used to calculate the aspect ratio. The maximum diameter of the lung-tuned antenna (511) based on the isocontour is about 1.2 cm, compared to about 0.70 cm in the liver-tuned antenna (512).

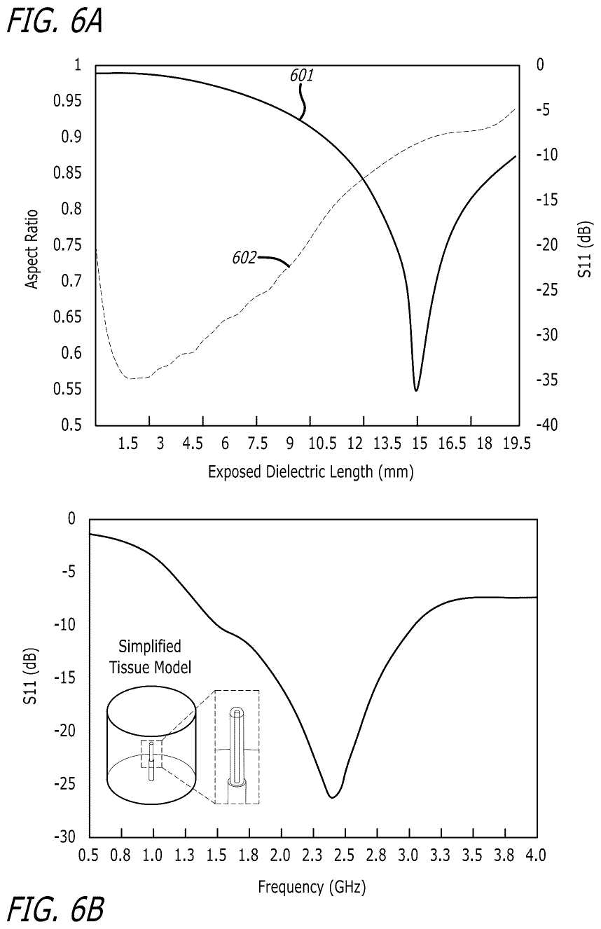

Many embodiments provide antenna optimization for monopole antenna designs. An optimal design for the lung and liver can be based on the optimization function (details about optimization function can be found in equation 9 in Exemplary Embodiments). In some embodiments, the global maximum may be at the exposed dielectric length of about 15.5 mm and about 13 mm, respectively. Several embodiments provide that the reflection coefficient can be very sensitive to the exposed dielectric length. A chart of the reflection coefficient (S₁₁) and aspect ratio as a function of increasing exposed dielectric length is illustrated in FIG. 6A in accordance with an embodiment of the invention. The reflection coefficient (S₁₁, 601) reaches a minimum value at about 15.5 mm exposed dielectric length when using a lung-tissue medium. Several embodiments implement the optimal monopole antenna length for minimizing the reflection coefficient. The aspect ratio (602) first decreases and starts to increase at around 1.5 mm exposed dielectric length. Beyond 1.5 mm exposed dielectric length, the aspect ratio of the monopole antenna increases in value with increasing exposed dielectric length. The aspect ratio (602) only increases minimally with each unit increase in length, while the reflection coefficient (601) increases by up to 10 dB for each millimeter increase in length.

A chart of reflection coefficient (dB) as a function of operating frequency of a tissue model in accordance with an embodiment is illustrated in FIG. 6B. An example of the tissue model used to calculate the reflection coefficient is also shown. The cost function can be utilized to weigh the benefit of each performance metric to provide the best organ-tuned antenna performance.

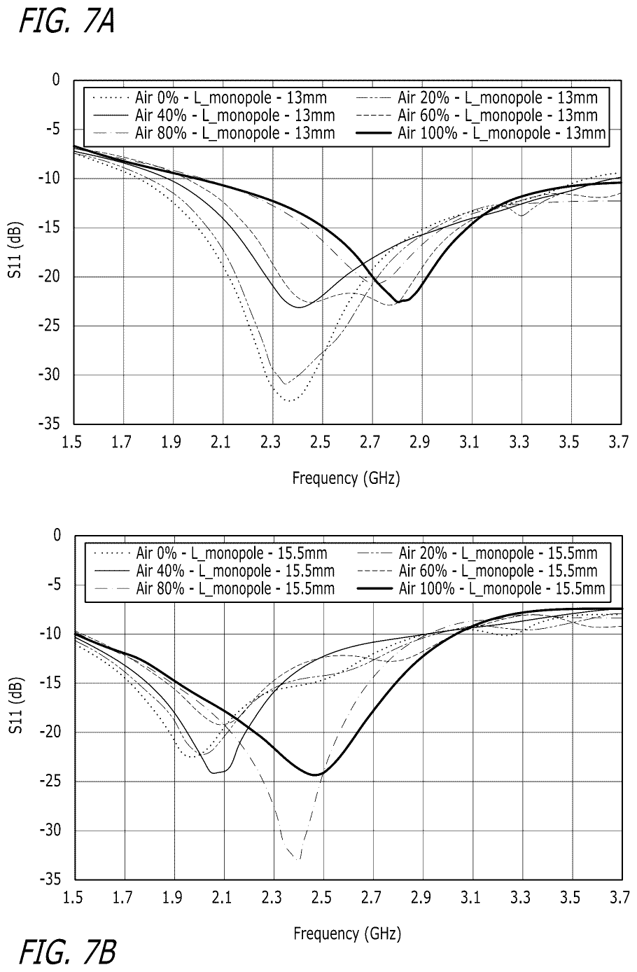

Several embodiments provide the relationship between air content percentage related to the constitutive parameters of the lung model. These parameters include (but are not limited to) the relative dielectric constant and the conductivity. In some embodiments, air content of about 0% represents the fully deflated state of the lung, and air content of about 100% represents the fully inflated state of the lung. Intermediate states with the lung model partially inflated can be evaluated using logistic regression modeling. A number of embodiments provide the resulting curves of the relative dielectric constant and conductivity calculated for air content varying from 0% to 100%. Full-wave simulations for 13.0 mm and 15.5 mm monopole antennas are carried out for each of the air content states in accordance with certain embodiments. Simulations of the S₁₁ parameter vs frequency for the 6 air content states utilizing the 13 mm and 15.5 mm monopole lengths in accordance with an embodiment are illustrated in FIGS. 7A and 7B respectively. FIG. 7B shows that 15.5 mm lung tuned monopole results in improved S₁₁ parameter for high air content (80% and 100% air filling). FIG. 7A shows that 13 mm liver tuned monopole performs better in a deflated lung compared to an inflated lung.

Several embodiments provide reflected power of both the 13.0 mm liver-tuned and 15.5 mm lung-tuned antennas in the ex vivo ventilator-inflated lung tissue. In some embodiments, both 13.0 mm and 15.5 mm antennas exhibit a monotonic increase in reflected power over time in both the 50 W and 100 W groups.

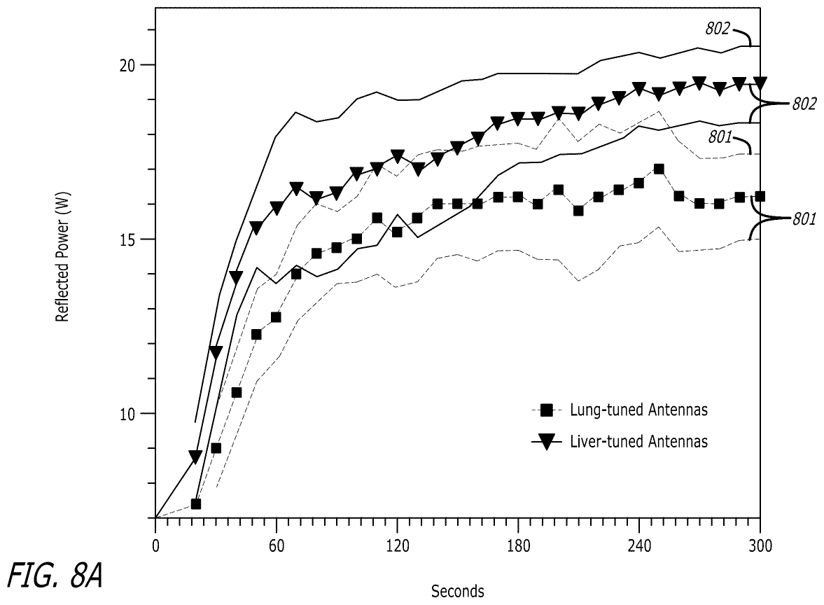

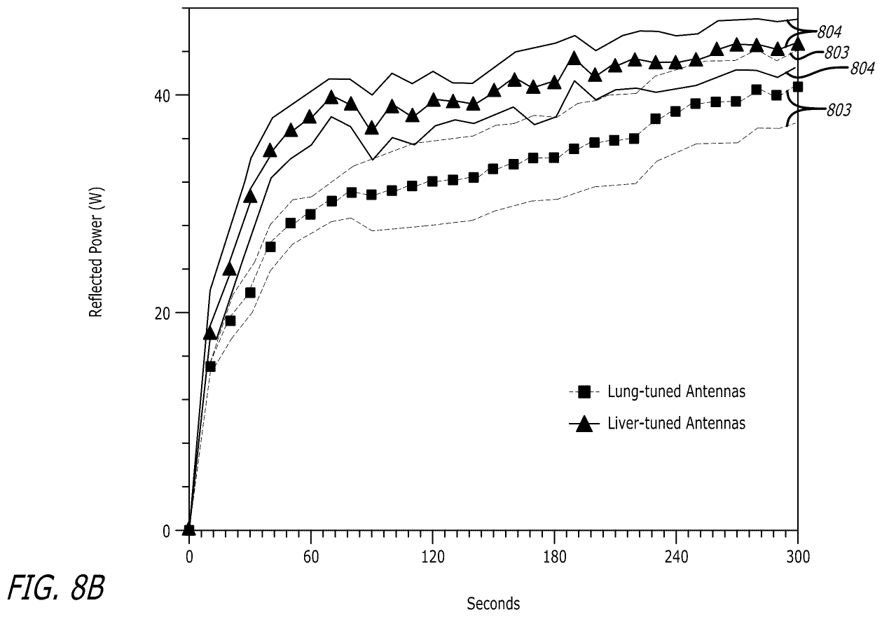

Reflected power at 2.45 GHz across lung-tuned antenna and liver-tuned antenna using the ex vivo ventilated porcine lung model at 50 W for 5 minutes is illustrated in FIG. 8A in accordance with an embodiment of the invention. The dashed lines specify the upper and lower bounds of the reflected power. The lung-tuned antenna (801) is more efficient in delivering microwave energy, recording less reflected power compared to the liver-tuned antenna (802). At 50 W, the lung-tuned antenna trends toward less reflection power at the 1 minute mark (mean±standard deviation, 12.7±3.1 W for lung tuned antenna vs 16.6±6.5 W for liver tuned antenna, p=0.33) and the 5 minute mark (16.2±2.8 W for lung tuned antenna vs 19.4±2.9 W for liver tuned antenna, p=0.04).

Reflected power at 2.45 GHz across lung-tuned antenna and liver-tuned antenna using the ex vivo ventilated porcine lung model at 100 W for 5 minutes is illustrated in FIG. 8B in accordance with an embodiment of the invention. The dashed lines specify the upper and lower bounds of the reflected power. The lung-tuned antenna (803) is more efficient in delivering microwave energy, recording less reflected power compared to the liver-tuned antenna (804). At 100 W, the lung-tuned antenna has less reflected power at the 1 minute mark (29.0±3.5 W for lung tuned antenna vs 38.0±5.3 W for liver tuned antenna, p=0.02). In the 50 W group, this difference in reflected power is more significant at both the 1 minute and 5 minutes while in the 100 W group, there is less power reflected only at the 1 minute time point.

Many embodiments provide microwave ablation sizes for lung tuned antennas and liver tuned antennas in the ex vivo ventilator-inflated lung tissue. Some embodiments show that in the 50 W cohort at 1 minute, the lung-tuned antennas, compared to the liver-tuned antennas, create a slightly larger but shorter ablation length (3.4±0.5 cm for lung tuned antennas vs 3.6±0.9 cm for liver tuned antennas, p=0.53). In several embodiments, the lung-tuned antennas have a larger ablation aspect ratio compared to the liver-tuned antenna (0.43±0.07 for lung tuned antennas vs 0.38±0.04 for liver tuned antennas, p=0.04). The lung-tuned antenna can create significantly more spherical ablation zones compared to the liver-tuned antenna at the 1 minute time point using 50 W in accordance with many embodiments.

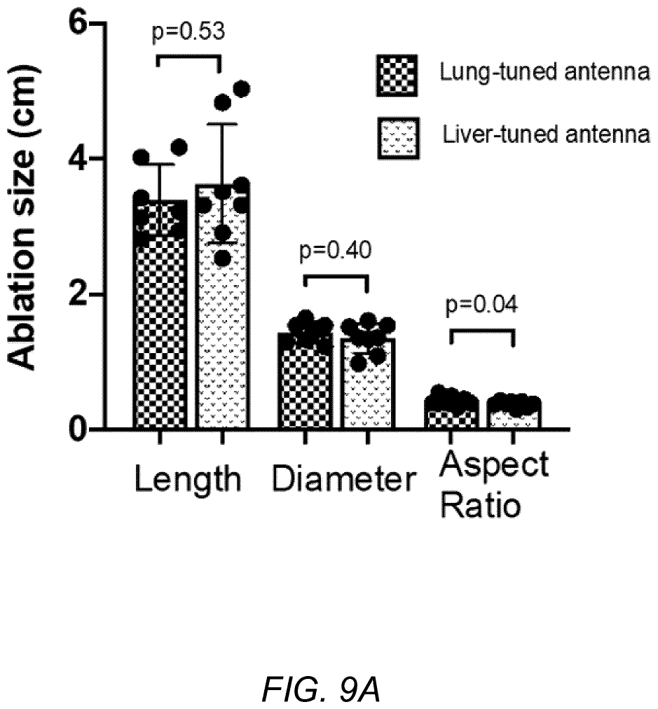

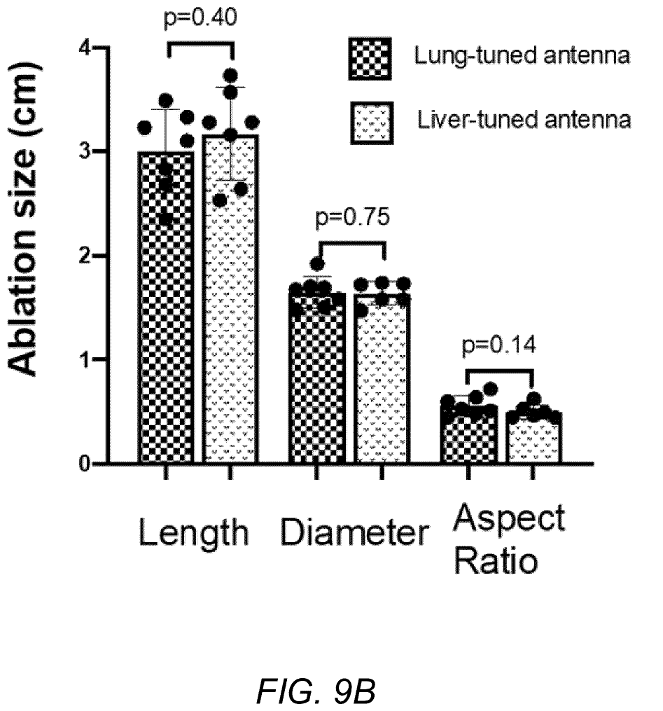

Microwave ablation size in lung tissue over a period of 1 and 5 minutes using 50 W power is illustrated in FIG. 9A in accordance with an embodiment of the invention. In the 50 W, 5 minute ablation group, the lung-tuned antennas create similar sized ablation zone widths (2.8±0.2 cm for lung tuned antennas vs 2.9±0.3 cm for liver tuned antennas, p=0.77) and ablation zone lengths (5.1±0.6 cm for lung tuned antennas vs 5.4±0.6 cm for liver tuned antennas, p=0.47), resulting in aspect ratios that are not significantly different from each other (0.56±0.06 for lung tuned antennas vs 0.54±0.09 for liver tuned antennas, p=0.74).

Microwave ablation size in lung tissue over a period of 1 and 5 minutes using 100 W power is illustrated in FIG. 9B in accordance with an embodiment of the invention. Within the samples that undergo 1 minute ablations at 100 W, the lung-tuned antennas create ablation zones with widths and lengths that are not significantly different from the liver tuned antennas (2.8±0.2 cm for lung tuned antennas vs 2.8±0.4 cm for liver tuned antennas, p=0.77 and 5.1±0.6 cm for lung tuned antennas vs 5.3±0.6 cm for liver tuned antennas, p=0.65, respectively). In the group that undergo 5 minutes ablations at 100 W, the lung-tuned antennas show similar-sized widths as the liver-tuned antennas (4.4±0.8 cm for lung tuned antennas vs 4.9±0.5 cm for liver tuned antennas, p=0.34) but have a shorter ablation zone length (5.4±0.5 cm for lung tuned antennas vs 6.9±0.8 cm for liver tuned antennas, p=0.01). As a result, the lung-tuned antennas create a more spherical ablation zone.

While characterization and performance of lung tuned monopole antennas are described above with reference to FIG. 5 to FIG. 9, any of a variety of monopole antenna that is tuned to match impedance of lung tissue can be utilized in the design and/or fabrication of tunable antenna as appropriate to the requirements of specific applications in accordance with various embodiments of the invention.

### Liquid Metal Implementation for Tunable Coaxial Antenna

Many embodiments implement liquid metal with a coaxial antenna to achieve tunable properties. In several embodiments, tunability of a coaxial antenna can be achieved with pressure-actuated liquid metal. Some embodiments implement pressure-actuated eutectic liquid metal. Examples of pressure-actuated eutectic liquid metal in such embodiments can include (but are not limited to): gallium-indium (EGaIn). As can readily be appreciated, any of a variety of types of liquid metal can be utilized as appropriate to the requirements of specific applications in accordance with various embodiments of the invention.

In many embodiments, liquid metal can be used in any part of a coaxial cable antenna that is conductive. Examples of conductive parts of a coaxial cable in accordance with various embodiments of the invention include (but are not limited to): inner conductor and/or outer conductor. The inner conductor can be physically augmented or connected to the metallic tip to add additional flexibility in impedance matching. In further embodiments, liquid metal can replace the outer conductor and enable tuning by changing the length of the exposed dielectric. As can readily be appreciated, any of a variety of conductive parts of a coaxial cable can be utilized as appropriate to the requirements of specific applications in accordance with various embodiments.

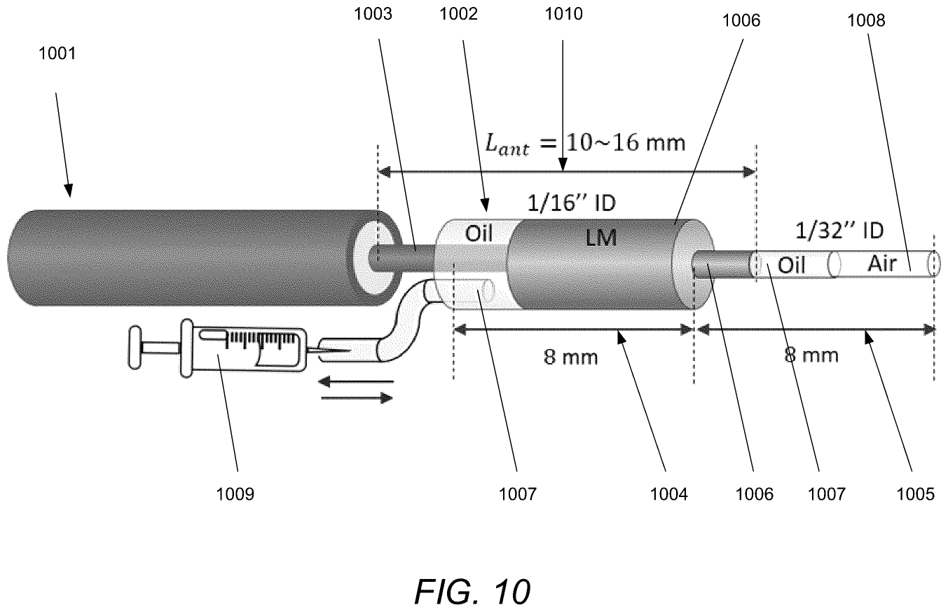

In some embodiments, EGaIn can be a liquid metal alloy at room temperature that shows low toxicity, high conductivity (about 3.4×10⁶ S/m) and good flexibility. FIG. 10 illustrates the design of a liquid metal actuated tunable coaxial monopole in accordance with an embodiment of the invention. A coaxial cable (1001) can be stripped or etched off with exposed inner conductor (1003) at the end and interconnected to a dielectric tube (1002). The coaxial cable can be a 086 coaxial cable. A section of the dielectric tube with about 1/16″ inner diameter (1004) can then be connected to another section of a dielectric tube with about 1/32″ inner diameter (1005). The section of the dielectric tube with about 1/16″ inner diameter (1004) can be about 8 mm in length. The section of a dielectric tube with about 1/32″ inner diameter (1005) can be about 8 mm in length. Examples of dielectric material include (but are not limited to) polytetrafluoroethylene (PTFE). As can readily be appreciated, any of a variety of dielectric material can be utilized as appropriate to the requirements of specific applications in accordance with various embodiments.

Liquid metal can be used as inner conductor and/or outer conductor. In some embodiments, liquid metal can replace the outer conductor of a coaxial antenna and enable tuning by changing the length of the exposed dielectric. The liquid metal (1006) can be partially filled within the tubes and passes across the interface. The remaining volume of the tubes can be filled with carrier liquid non-conductive oil (1007), with optical transparency and low loss in radio frequency (with tan δ<0.01). In certain embodiments, the non-conductive oil can be mineral oil.

Since the inner connector can be in direct contact with the liquid metal plug, the total monopole length (Lₐₙₜ) (1010) can be measured from the end of the outer conductor to the interface between liquid metal and oil. The length of the monopole antenna can be tuned to match impedance of an targeted organ including (but not limited to): liver, lung, pancreas, spleen, adrenal gland, kidney, bladder, uterus, prostate, and/or bone to reduce reflected power. The monopole length can be from about 10 mm to about 16 mm. The length of the monopole antenna in accordance with certain embodiments can be tuned for an inflated lung and the length is about 15.5 mm. In several embodiments, the operating frequency of the monopole antenna is from about 915 MHz to about 8GHz. Some embodiments operates at the frequency of about 2.45 GHz. In many embodiments, the incident power of the monopole antenna is from about 20 W to about 200 W. Several embodiments use the incident power of about 50 W or about 100 W.

In several embodiments, liquid metal antenna tuning can be achieved by a pneumatic-driven physical displacement of liquid metal plug inside a dielectric tube interconnecting with the coaxial cable. Some embodiments include pumping a liquid metal through the inner metal to the tip of the antenna such that it can provide a weighted load that can augment the impedance matching process. In some other embodiments, digital tuning methods can be adopted. Many embodiments use a matching circuit comprised of several discrete inductors and capacitors. The matching circuit may adjust the antenna output impedance to match the target biological tissue impedance to optimize heating. In some embodiments, liquid metal antenna tuning can be achieved by an electrical-driven physical displacement of liquid metal plug inside a dielectric tube interconnecting with the coaxial cable. Examples of electrical-driven approach in accordance with various embodiments of the invention include (but are not limited to): DC voltage differences applied through a carrier electrolyte—utilizing the effect of electro-wetting. The section of air (1008) sealed at the end of the 1/32″ tube may provide a reversible tuning range of a few millimeter, that can be pneumatic driven by (but not limited to) syringe, pneumatic pump, or pressure regulator (1009). The liquid metal coaxial monopole could therefore operate with a tunable length to achieve the optimal impedance matching under varying tissue dielectric properties.

While various structures for tunable coaxial antenna using liquid metal are described above with reference to FIG. 10, any of a variety of structures that utilize liquid metal with coaxial antenna to match impedance of different tissue can be utilized in the design and/or fabrication of tunable antenna as appropriate to the requirements of specific applications in accordance with various embodiments of the invention.

### Cooling Mechanism Implementation for Tunable Coaxial Antenna

Many embodiments implement a cooling mechanism with a coaxial antenna to achieve a cooling effect during impedance matching. In several embodiments, cooling of a coaxial antenna can be achieved by introducing water and/or gas around the shaft of the coaxial cable. The water and/or gas can be localized to the coaxial cable by having tight seal around a metallic cannula, ceramic and metallic tip. Some embodiments implement metallic tips attached to ceramic layer of coaxial cable with or without direct connection to the inner metal conductor, which may include parts of the liquid metal. In several embodiments, a tunable coaxial antenna with cooling mechanism can minimize shaft heating during impedance mismatches.

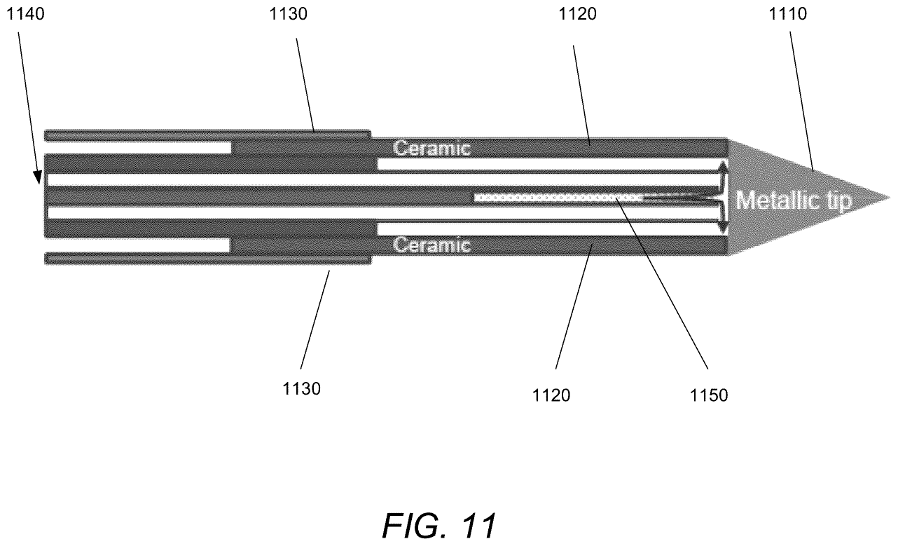

FIG. 11 illustrates the design of a liquid metal actuated tunable coaxial antenna with metallic tip in accordance with an embodiment of the invention. The coaxial cable antenna 1140 has a dimeter of about 0.020″. A PTFE tube with diameter of about 0.017″ is located inside the coaxial cable (1140). Liquid metal (1150) can be partially filled in the PTFE tube. Steel outer sheath (1130) surrounds the coaxial cable (1140). Ceramic layers (1120) sandwich in between steel outer sheath and the coaxial cable. A metallic tip (1110) can be attached to the ceramic layers.

While various structures for tunable coaxial antenna incorporating cooling mechanism are described above with reference to FIG. 11, any of a variety of structures that can cool liquid metal coaxial antenna during impedance matching process of different tissue can be utilized in the design and/or fabrication of tunable antenna as appropriate to the requirements of specific applications in accordance with various embodiments of the invention.

### EXEMPLARY EMBODIMENTS

The following embodiments provide specific optimization of structures and geometries of monopole antennas that enable tuning in organs and/or tissues during microwave ablation processes. Many embodiments implement antennas with geometries tuned to various organs including (but not limited to) lung and liver. It will be understood that the specific embodiments are provided for exemplary purposes and are not limiting to the overall scope of the disclosure, which must be considered in light of the entire specification, figures and claims.

### Lung-Tuned Monopole Antenna for Microwave Ablation

Many embodiments provide optimized monopole antennas to transmit energy efficiently into the dielectric load of an air expanded lung while maintaining a spherical ablation zone. This lung-tuned antenna in accordance with several embodiments can be used to create microwave ablation zones in a ventilator-controlled ex vivo porcine lung model. Some embodiments provide analytic modeling and antenna optimization of monopole antenna designs that can efficiently deliver microwave energy into lung tissue.

### Analytical Modeling of Lung Tuned Antenna Designs

Microwave ablation antennas can utilize semi-rigid copper coaxial cables to act as the transmission line between the power generator and the target tissue. The impedance load of the microwave antenna can be modulated by changing the geometry at the end of the coaxial cable in accordance with some embodiments. The monopole antenna is one of the most widely used class of antennas and its general geometry can be constructed by stripping off a section of outer conductor at the end of coaxial cable. The dimensions of the 0086 (0.085″) copper coaxial cable are listed in Table 2. The length of the underlying λ_{eff} represents the effective wavelength for the design of the coaxial antenna. The structure of a coaxial monopole antenna is illustrated in FIG. 3A.

The optimal exposed dielectric length can be analytically calculated by evaluating the insulating antenna as an inner conductor wrapped within multiple dielectric layers and immersed in a lossy dielectric medium. This methodology treats the insulating antenna as multiple sections of a lossy transmission line with generalized propagation constants to account for both ohmic losses as well as radiative losses from the antenna to the ambient medium. The multi-layered insulated antenna can then be represented by a multi-sectioned transmission line, with each section terminated with a load impedance equivalent to the input impedance of the preceding section. (See, e.g., R. W. King, IEEE Transactions on Antennas and Propagation, 1964, 12, 3, 305-318; the disclosure of which is incorporated herein by reference.) In the case of the monopole design, the insulating monopole antenna consists of a central conductive cylinder with radius a and length h (representing the inner conductor of coaxial cable, FIG. 3A), wrapped by cylindrical dielectric region with radius b and dielectric constant ϵ₂ (representing the Teflon filled in coaxial cable, region 2 in FIG. 3A), immersed in infinite homogeneous medium with dielectric constant ϵ₃ and conductivity σ₃ (region 3 in FIG. 3A). In certain embodiments, the electrical properties of an inflated lungs are utilized as a surrogate variable to account for the presence of air that is present within the lungs.

The wavenumber in the insulating and lossy dielectric regions is therefore:

k₂=ω\sqrt{(μ₀ϵ₂)},k₃=ω{square root over (μ₀ϵ₃−jσ₃/ω))} (2)

where μ₀ is the free space permeability assumed to apply in all regions. An e^{jωt} time convention is applied. This approach also may require that the wavenumber in ambient medium is greater than that of the insulating dielectric, and that the cross section of the antenna is electrically small:

|k₃/k₂|²>>1,(k₂b)²<<1 (3)

Assuming the antenna is excited with voltage source V₀^{e}, the current carried by the antenna central conductor is thus given as:

$$I ( z ) = I ( 0 ) \frac{sin {k}_{eff} ( h - \vert z \vert )}{sin {k}_{eff} h} = j \frac{{V}_{0}^{e}}{2 {Z}_{e}} \frac{sin {k}_{eff} ( h - \vert z \vert )}{cos {k}_{eff} h} \qquad ( 4 )$$

where I(0) represents the current at z=0. The characteristic impedance Z_{c} and the effective propagation constant k_{eff} are given by:

$${Z}_{e} = \frac{ω {μ}_{0} {k}_{eff}}{2 π {k}_{2}^{2}} [ ln \frac{b}{a} + \frac{{k}_{2}^{2}}{{k}_{3}^{2}} \frac{{H}_{0}^{( 1 )} ( {k}_{3} b )}{{k}_{3} {bH}_{1}^{( 1 )} ( {k}_{3} b )} ] \qquad ( 5 )$$

$${k}_{eff} = {{k}_{2} [ \frac{ln ( \frac{b}{a} ) {k}_{3} {bH}_{1}^{( 1 )} ( {k}_{3} b ) + {H}_{0}^{( 1 )} ( {k}_{3} b )}{ln ( \frac{b}{a} ) {k}_{3} {bH}_{1}^{( 1 )} ( {k}_{3} b ) + \frac{{k}_{2}^{2}}{{k}_{3}^{2}} {H}_{0}^{( 1 )} ( {k}_{3} b )} ]}^{1 / 2} \qquad ( 6 )$$

$${k}_{eff} = {β}_{eff} + j {α}_{eff} \qquad ( 7 )$$

where H⁽¹⁾ represents Hankel function of the first kind. To corroborate its validity, it can be readily verified that in a case where k₃ approaches to k₂, the effective propagation constant k_{eff} also approaches to k₂, reducing the case to a monopole immersed in dielectric material. The real part of the effective propagation constant β_{eff} can then be directly linked to the effective wavelength

$${λ}_{eff} = \frac{2 π}{{β}_{eff}} ,$$

from which the optimal exposed dielectric length of monopole antenna can be determined.

### Optimization of Lung Tuned Antennas

Criteria for optimizing microwave ablation antennas may include small diameter for ease of percutaneous insertion, good impedance matching for efficient power transmission, and localized spherical ablation pattern for more precise ablation treatment. Several embodiments implement a computational parametric approach to find the optimal ablation design, weighing not only the reflection coefficient but also the shape of the ablation zone. The specific absorption rate (SAR) can be calculated for various lengths of the exposed monopole dielectric in the vicinity of the optimal length. These lengths may be altered in 0.5 mm intervals as fabrication tolerance. Simulations can be performed in a two dimensional domain assuming a rotational symmetry on the longitudinal axis of the antenna. Analysis can be performed using finite-element approach to solve Maxwell's equations in the transverse magnetic (TM) propagation mode (COMSOL Multiphysics 5.5):

$${∇}^{2} E = - {ω}^{2} {μ}_{0} ( {ϵ}_{0} {ϵ}_{r} ( f ) - j \frac{σ ( f )}{ω} ) E \qquad ( 8 )$$

where E is the electric field vector (V/m), ω is the angular frequency (rad/sec), ϵᵣ is the relative permittivity and σ is the effective conductivity (S/m). Lung and liver properties from Table 1 can be utilized to visualize the SAR map. To create the most spherical ablation zone, an aspect ratio (AR) from the SAR map can be calculated, which can be defined as twice the maximum radial dimension (diameter) divided by the longitudinal dimension (length) of the SAR=300 W/kg isocontour in the SAR maps.

An optimization function Ψ can be utilized to find the antenna that balanced antenna impedance matching at 2.45 GHz in terms of S₁₁ (in dB scale) with the most spherical ablation zone, characterized by the highest AR. Ψ can be defined as:

$$Ψ ( {S}_{11} , AR ) = \frac{1}{1 + {e}^{{A}_{1} ( {S}_{11} + {B}_{1} )}} + \frac{1}{1 + {e}^{{A}_{2} ( AR + {B}_{2} )}} \qquad ( 9 )$$

where A₁ and A₂ represent slopes of the sigmoidal curves at inflection points B₁ and B₂. In general, reflection coefficients greater than −20 dB or heating aspect ratios less than 0.7 are considered too inefficient or too elongated of an ablation zone, respectively. With these constraints, the coefficient values selected for the above equation are A₁=0.7, A₂=20, B₁=−15 and B₂=0.7. This equation can be adapted for various organs, based on expected S₁₁ and aspect ratio. The optimization function may be able to provide discriminatory value in filtering out antennas that are too elongated in shape or had too high of a reflection coefficient. A propensity score can be calculated for each permutation in the parametric analysis, with the highest score indicating the most optimal design that balances reflected power against most spherical ablation zone design.

### Experimental Setup

Many embodiments construct lung-tuned and liver-tuned monopole antennas from polytetrafluorethylene (PTFE) filled, semi-rigid coaxial cable (UT-085C; Micro-Coax LLC) with exposed dielectric lengths calculated from the analytical solution. An industrial high-powered solid state 2.45 GHz microwave generator (Sairem) can be used to create the ablation zones.

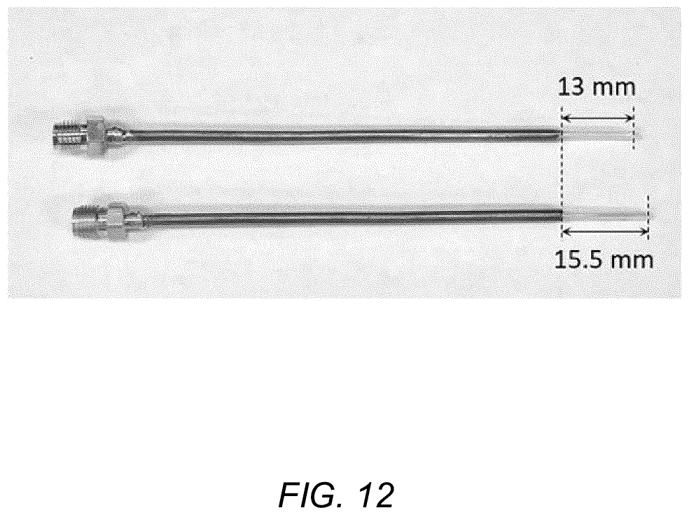

Several embodiments perform microwave ablation experiments on freshly excised ex vivo porcine lung tissues with attached airways. The lungs are removed en-bloc and allowed to reach room temperature (about 25° C.) over a period of 1-2 hours. The lung tissue can be mechanically ventilated to maintain a 600 mL volume controlled airspace to mimic a fully expanded lung. The lung tuned 15.5 mm antennas can be inserted 5 cm into the central portions of the upper, middle and lower fields of the right lung. Microwave ablations are created at about 50 W and about 100 W for about 1 minute and about 5 minutes with reflected power being recorded in 10-second intervals from the microwave generator display. Corresponding matched ablations can be then created in the left lung with the liver-tuned 13.0 mm monopole antennas as controls using the same power and time settings. Sample images of the two antennas are shown in FIG. 12. Ablation zones (n=7 for each power/time combination) are created in the ex vivo lung tissue using the lung-tuned antenna and liver-tuned antenna.

After the ablations are completed, ablation zones can be sectioned across their insertion path, revealing a cross section of the ablation zone. The congestive component of the ablation zone can be measured and served as the outer border of the ablation zone. Ablation length and width are recorded and compared between antennas. Student t-test can be performed to evaluate differences in each ablation zone metric. P-values less than 0.05 are considered significant.

### DOCTRINE OF EQUIVALENTS

As can be inferred from the above discussion, the above-mentioned concepts can be implemented in a variety of arrangements in accordance with embodiments of the invention. Accordingly, although the present invention has been described in certain specific aspects, many additional modifications and variations would be apparent to those skilled in the art. It is therefore to be understood that the present invention may be practiced otherwise than specifically described. Thus, embodiments of the present invention should be considered in all respects as illustrative and not restrictive.

What is claimed is:

1. A tunable monopole antenna comprising:
a coaxial cable comprising an inner conductor and an outer conductor, wherein a first length of one end of the coaxial cable has the outer conductor removed and the inner conductor exposed;
wherein the first length of the exposed inner conductor is not greater than an effective quarter wavelength (λ_{eff}/4) of the coaxial antenna;
a first dielectric tube, wherein the first dielectric tube has a diameter and connects to the exposed inner conductor along a center of the coaxial cable;
wherein the first dielectric tube covers a second length of the exposed inner conductor;
wherein a first part of the first dielectric tube is filled with a liquid metal, and a second part of the first dielectric tube is filled with a non-conductive oil;
wherein the first dielectric tube is connected with a pressure regulator;
a second dielectric tube, wherein the second dielectric tube has a diameter and connects to the first dielectric tube along a center of the first dielectric tube;
wherein a first part of the second dielectric tube is filled with a liquid metal, and a second part of the second dielectric tube is filled with mineral oil; and
wherein the length of the monopole antenna is from the exposed end of the outer conductor to the interface between the liquid metal and mineral oil of the second dielectric tube.

2. The tunable monopole antenna of claim 1, wherein the length of the monopole antenna is tunable by applying a pressure to the pressure regulator.

3. The tunable monopole antenna of claim 1, wherein the length of the monopole antenna is tunable and the tunable length is from about 10 mm to about 16 mm.

4. The tunable monopole antenna of claim 1, wherein the length of the monopole antenna is tuned for an inflated lung and the length is about 15.5 mm.

5. The tunable monopole antenna of claim 1, wherein an operating frequency of the tunable monopole antenna is from about 915 MHz to about 8GHz.

6. The tunable monopole antenna of claim 5, wherein the operating frequency is about 2.45 GHz.

7. The tunable monopole antenna of claim 1, wherein an incident power of the tunable monopole antenna is from about 20 W to about 200 W.

8. The tunable monopole antenna of claim 7, wherein the incident power is about 50 W or about 100 W.

9. The tunable monopole antenna of claim 1, wherein the second length of the exposed inner conductor covered by the first dielectric tube is smaller than the first length of the exposed inner conductor.

10. The tunable monopole antenna of claim 1, wherein the diameter of the second dielectric tube is smaller than the diameter of the first dielectric tube.

11. The tunable monopole antenna of claim 1, wherein the diameter of the first dielectric tube is about 1/16 inch.

12. The tunable monopole antenna of claim 1, wherein the diameter of the second dielectric tube is about 1/32 inch.

13. The tunable monopole antenna of claim 1, wherein the length of the first dielectric tube is about 8 mm.

14. The tunable monopole antenna of claim 1, wherein the length of the second dielectric tube is about 8 mm.

15. The tunable monopole antenna of claim 1, wherein the dielectric tube comprises polytetrafluoroethylene.

16. The tunable monopole antenna of claim 1, wherein the liquid metal is a pressure-actuated eutectic liquid metal.

17. The tunable monopole antenna of claim 1, wherein the liquid metal comprises gallium-indium.

18. The tunable monopole antenna of claim 1, wherein the pressure regulator is a syringe.

19. The tunable monopole antenna of claim 1, wherein the non-conductive oil is mineral oil.

20. The tunable monopole antenna of claim 1, further comprising a metallic tip.

21. The tunable monopole antenna of claim 20, wherein the metallic tip is fitted within a ceramic shaft.

22. A method for performing microwave ablation comprising:
providing a power source;
providing a tunable monopole antenna comprising:
a coaxial cable comprising an inner conductor and an outer conductor, wherein a first length of one end of the coaxial cable has the outer conductor removed and the inner conductor exposed;
wherein the first length of the exposed inner conductor is not greater than an effective quarter wavelength (λ_{eff}/4) of the coaxial antenna;
a first dielectric tube, wherein the first dielectric tube has a diameter and connects to the exposed inner conductor along a center of the coaxial cable;
wherein the first dielectric tube covers a second length of the exposed inner conductor;
wherein a first part of the first dielectric tube is filled with a liquid metal, and a second part of the first dielectric tube is filled with a non-conductive oil;
wherein the first dielectric tube is connected with a pressure regulator;
a second dielectric tube, wherein the second dielectric tube has a diameter and connects to the first dielectric tube along a center of the first dielectric tube;
wherein a first part of the second dielectric tube is filled with a liquid metal, and a second part of the second dielectric tube is filled with mineral oil; and
wherein the length of the monopole antenna is from the exposed end of the outer conductor to the interface between the liquid metal and mineral oil of the second dielectric tube;
inserting the tunable monopole antenna to a target organ;
applying an incident power to the target organ, wherein the incident power has an operating frequency.

23. The method of claim 22, wherein the length of the monopole antenna is tunable by applying a pressure to the pressure regulator.

24. The method of claim 22, wherein the length of the monopole antenna is tuned to match the target organ impedance during microwave ablation to lower reflect power.

25. The method of claim 24, wherein the length of the monopole antenna is tuned to match the impedance of a deflated lung, an inflated lung, or a liver.

26. The method of claim 22, wherein the length of the monopole antenna is tunable and the tunable length is from about 10 mm to about 16 mm.

27. The method of claim 22, wherein the length of the monopole antenna is tuned for an inflated lung and the length is about 15.5 mm.

28. The method of claim 22, wherein the operating frequency is from about 915 MHz to about 8 GHz.

29. The method of claim 28, wherein the operating frequency is about 2.45 GHz.

30. The method of claim 22, wherein the incident power is from about 20 W to about 200 W.

31. The method of claim 30, wherein the incident power is about 50 W or about 100 W.

32. The method of claim 22, wherein the second length of the exposed inner conductor covered by the first dielectric tube is smaller than the first length of the exposed inner conductor.

33. The method of claim 22, wherein the diameter of the second dielectric tube is smaller than the diameter of the first dielectric tube.

34. The method of claim 22, wherein the diameter of the first dielectric tube is about 1/16 inch.

35. The method of claim 22, wherein the diameter of the second dielectric tube is about 1/32 inch.

36. The method of claim 22, wherein the length of the first dielectric tube is about 8 mm.

37. The method of claim 22, wherein the length of the second dielectric tube is about 8 mm.

38. The method of claim 22, wherein the dielectric tube comprises polytetrafluoroethylene.

39. The method of claim 22, wherein the liquid metal is a pressure-actuated eutectic liquid metal.

40. The method of claim 22, wherein the liquid metal comprises gallium-indium.

41. The method of claim 22, wherein the pressure regulator is a syringe.

42. The method of claim 22, wherein the non-conductive oil is mineral oil.

43. The method of claim 22, wherein the tunable monopole antenna further comprising a metallic tip.

44. The method of claim 43, wherein the metallic tip is fitted within a ceramic shaft.
